(19) United States
(12) Patent Application Publication
(10) Pub. No.: US 2009/0264710 A1
(43) Pub. Date: Oct. 22, 2009

(54) SURGICAL APPARATUS

(75) Inventors: Gursharan Singh Chana, West Midlands (GB); Christopher John Sloss, South Yorkshire (GB)

Correspondence Address: PATENTTM.US, P. O. BOX 82788, PORTLAND, OR, 97282-0788, US

(73) Assignee: COMIS ORTHOPAEDICS LIMITED, Rotherham (GB)

(21) Appl. No.: 12/296,925

(22) PCT Filed: Apr. 30, 2007
(86) PCT No.: PCT/GB2007/001570

§ 371 Date: Feb. 10, 2009

(30) Foreign Application Priority Data
Apr. 29, 2006 (GB) 0608561.7

(51) Int. Cl. A61B 1/32

(52) U.S. Cl. 600/233

(57) ABSTRACT

A surgical apparatus (10) comprises a support member (12), and first and second mounting arrangements (26, 28) for mounting respective first and second retractors (22, 24) mounted on the support member. The mounting arrangements are mounted on the support member, and at least the first mounting arrangement is movable towards and away from the second mounting arrangement.

This invention relates to surgical apparatus. More particularly, but not exclusively the invention relates to surgical apparatus for carrying out minimally invasive surgery. Embodiments of the invention relate to surgical apparatus for carrying out hip surgery, such as minimally invasive hip surgery.

Retraction systems for carrying out hip surgery, particularly minimally invasive hip surgery, require the use of significant force on the retractors to expose the acetabulum and femur, to allow for their preparation, and for the implantation of a prosthesis. In addition, some systems require assistance to hold the retractors in position.

According to one aspect of this invention there is provided a surgical apparatus comprising a support member, and first and second retractors mounted on the support member, wherein at least the first retractor is movable towards and away from the second retractor.

According to another aspect of this invention there is provided a surgical apparatus comprising a support member, and first and second mounting arrangements for mounting respective first and second retractors mounted on the support member, wherein the mounting arrangements are mounted on the support member, and at least the first mounting arrangement is movable towards and away from the second mounting arrangement.

The first retractor or mounting arrangement may be linearly movable towards and away from the second retractor or mounting arrangement. The first retractor or mounting arrangement may be pivotally movable relative to the support member. The second retractor or mounting arrangement may be pivotally movable relative to the support member.

The first and second retractors or mounting arrangements may be arranged in opposed positions relative to one another on the support member. Desirably, the first and second retractors or mounting arrangements face each other.

The support member may define an access region through which surgery can be carried out. The support member may surround the aforesaid access region. The retractors may extend through the access region. The support member may comprise first and second opposite sides upon which the first and second retractors or mounting arrangements are respectively mounted.

Each of the first and second sides may comprise a central region and opposite end regions extending transverse to the central region. The opposite end regions of each of the first and second sides may be angled relative to the central region at an angle in the range of 20° to substantially 30° to the central region. The opposite end regions may be angled substantially 30° to the central region. This provides the advantage in one embodiment that the support member conforms generally to the region of the body upon which the surgery is carried out.

The support member may comprise opposite third and fourth sides. The third and fourth sides may extend between the first and second sides to connect the first side to the second side, thereby providing a generally rectangular profile of the support member. In one embodiment, the support may have a generally square configuration.

The third and fourth sides may each include a movable securing member to secure a respective surgical member thereto. The third and fourth sides may include serrations to hold the securing member in a selected position on the respective third or fourth side. Each securing member may include a securing formation to which a connecting element can be attached to connect the respective surgical member to the securing member. The securing formation may comprise an upstanding projection. The connecting element may be elongate and may be flexible. In one embodiment, the connecting element may comprise a chain. Each surgical member may comprise a Hohman retractor.

The first and second retractors may be provided with a respective manipulating member to move the first and second retractors to the desired orientations.

Each surgical member may comprise a respective manipulating member to move the respective surgical member to its desired position. Each manipulating member may comprise a handle.

First and second mounting arrangements may be provided to mount respectively the first and second retractors on the support member. The first and second mounting arrangements may be fixedly mounted on the support member, for example by welding, riveting or by casting.

Each of the first and second mounting arrangements may comprise a securing assembly to secure the respective first and second retractor thereto. The securing assembly may comprise a fixed member and a fastener extending therethrough. The fastener may comprise a shaft, having a threaded region, and a head. The head may be provided at one end of the shaft. The threaded region of the shaft may be provided at the opposite end region of the shaft. The fastener may comprise a bolt.

Each of the first and second mounting arrangements may further include a movable member. The fastener may co-operatively engage the movable member. The movable member may be threaded to threadably engage the fastener.

The movable member may have interlocking formations. The fixed member may have corresponding interlocking formations to co-operatively engage the interlocking formations on the movable member. Thus, when the movable member is engaged on the fastener, the interlocking formations on the movable member may engage the interlocking formations on the fixed member.

In one embodiment, when the fastener tightens the movable member against the fixed member, the interlocking formations prevent relative rotation of the movable member and the fixed member.

Urging means may be provided to urge the movable member into the engagement with the fixed member. The urging means may comprise a spring, such as a coil spring.

The movable member may include a securing formation to secure the respective first or second retractor onto the movable member. The first and second retractors may each comprise a securing element to engage the respective movable member. The securing element may be slidably mounted on the securing formation. The securing formation and the securing element may be correspondingly configured to substantially prevent relative rotation of the movable member and the respective retractor.

In one embodiment, the securing formation may comprise a generally rectangular outer profile of the movable member. The securing element may have a generally rectangular inner profile, said inner profile of the securing element corresponding to the outer profile of the securing formation.

Each of the movable members may be pivotally movable relative to the respective first or second fixed members to arrange the respective first or second retractors at a desired angle relative to the support member.

The threaded engagement of the fastener with the respective movable member may lock the movable member against the fixed member, thereby preventing pivoting of the respective first or second retractor.

The first mounting arrangement may comprise a displacement mechanism to move the first retractor towards and away from the second retractor. The displacement mechanism may comprise a displacement member to which the respective fixed member is attached.

The displacement member may be elongate and may comprise a rack. The displacement mechanism may comprise a housing, through which the displacement member movably extends.

The first mounting arrangement may include a driver to drive the displacement member. The driver may comprise a rotatable head and a cog in engagement with the displacement member. The displacement member may comprise a plurality of teeth arranged to co-operate with the cog. The displacement member and the cog may comprise a rack and pinion arrangement.

A locking member may be provided to lock the displacement member to the housing. In a first embodiment, the locking member may comprise a threaded member threadably receivable in the housing to engage the mounting member. The locking member may further include a rotatable head mounted on the threaded member to rotate the threaded member.

In a second embodiment, the locking member may comprise a rocker assembly, which may comprise a depressible element for engaging the displacement members. The depressible element may be movable between a locking position and a release position by pressing thereon.

The depressible element may pivot about a pivot member between the locking and the release positions.

The depressible element may be capable of being pressed at a first region on one side of the pivot member to move the depressible element to the locking position.

The depressible element may be capable of being pressed at a second region on the opposite side of the pivot member to move the depressible element to the release position.

The rocker assembly may comprise an urging arrangement to urge the depressible element to the locking position. The urging arrangement may comprise a spring, such as a leaf spring. The urging arrangement may be provided on the depressible element, and may engage the mounting arrangement.

The mounting arrangement may include a reaction portion which the urging arrangement can engage. The urging arrangement may produce a reaction against the reaction portion when the depressible element is moved to the release position. The urging arrangement may react against the reaction portion to urge the depressible element to the locking position.

The surgical apparatus may include the first and second retractors mounted on the support member by the first and second mounting arrangements respectively.

According to another aspect of this invention, there is provided a method of using a surgical apparatus as described above, said method comprising arranging the first and second retractors on the member, inserting the retractors in an incision in a body and moving the first retractor towards and/or away from the second retractor to retract a portion of the body upon which surgery is being effected.

The method may comprise linearly moving the first retractor towards and/or away from the first retractor.

The method may comprise pivoting the first retractor relative to the support member. The method may comprise pivoting the second retractor relative to the support member.

The method may comprise arranging the first and second retractors in opposed positions relative to each other. The method may comprise arranging the first and second retractors such that they face each other.

The support member may define an access region, and the method may comprise carrying out the surgery through the access region.

The method may comprise mounting the retractors on first and second opposite sides of the support member.

In one embodiment, the method may comprise securing the surgical members to a respective one of third and fourth opposite sides of the support member. Each surgical member may comprise a Hohman retractor.

According to another aspect of this invention there is provided a retractor comprising a securing element for securing the retractor to a support member and a blade extending from the securing element.

The securing element may comprise a generally rectangular profile for co-operation with a correspondingly shaped securing part on the support member. The rectangular profile may be an inner rectangular profile.

The blade may have a longitudinal axis and may be curved about the longitudinal axis.

Embodiments of the invention will now be described by way of example only, with reference to the accompanying drawings, in which:

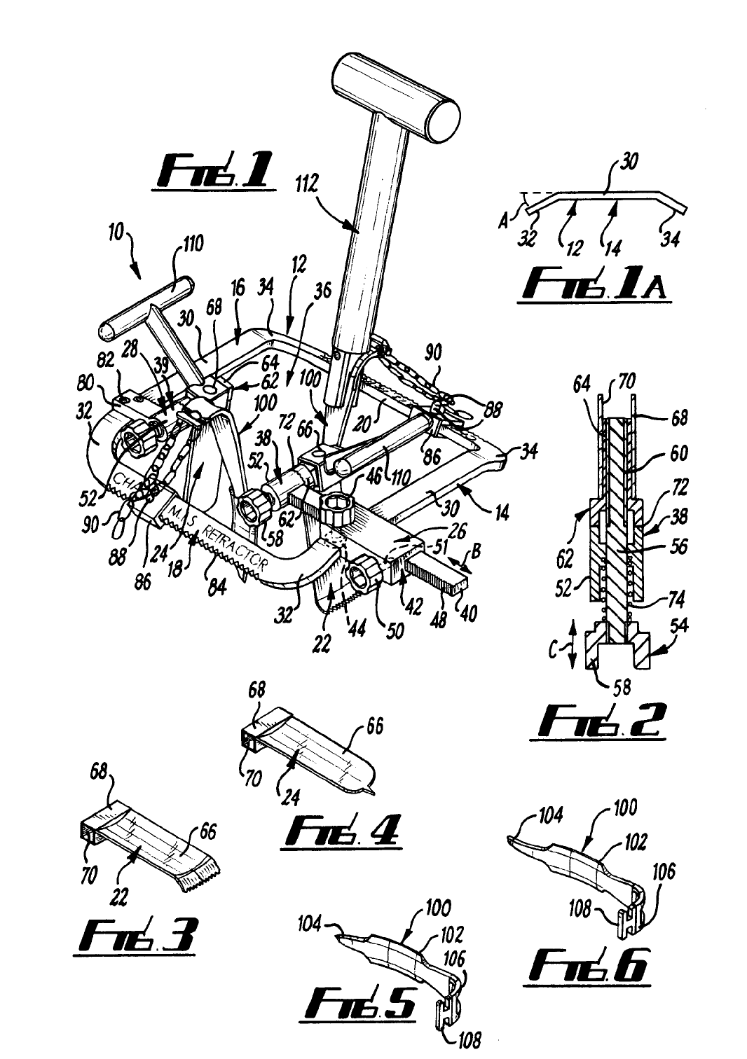

FIG. 1 is a perspective view of a first embodiment of a surgical apparatus;

FIG. 1A is a side view of a support member forming part of the surgical apparatus shown in FIG. 1;

FIG. 2 is a sectional view of a mounting arrangement forming part of the surgical apparatus shown in FIG. 1;

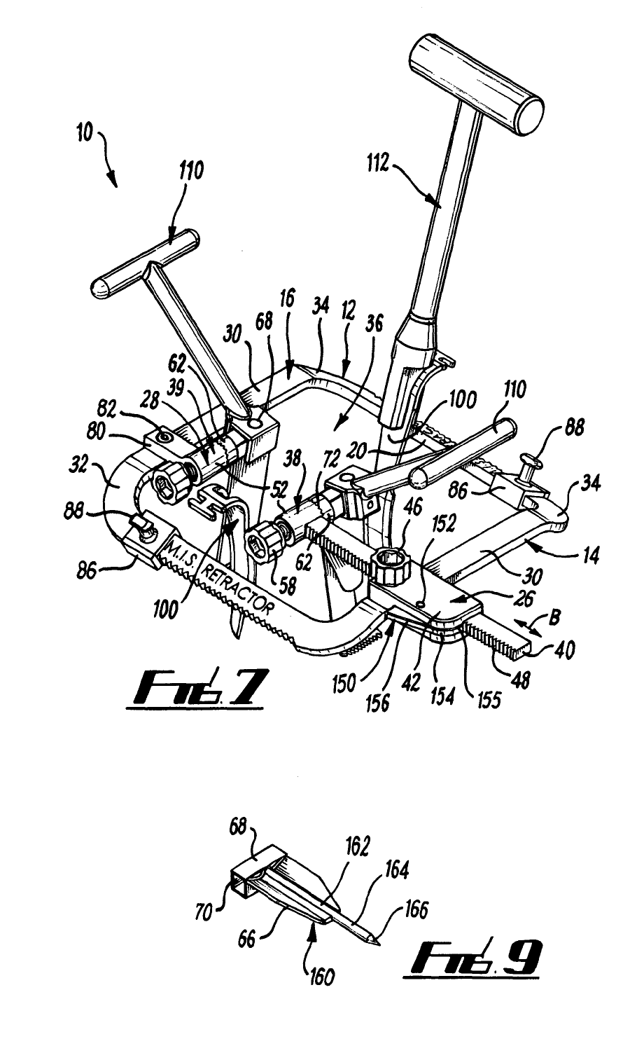

FIGS. 3 and 4 are perspective views of two retractors;

FIGS. 5 and 6 are perspective views of first and second Hohman retractors;

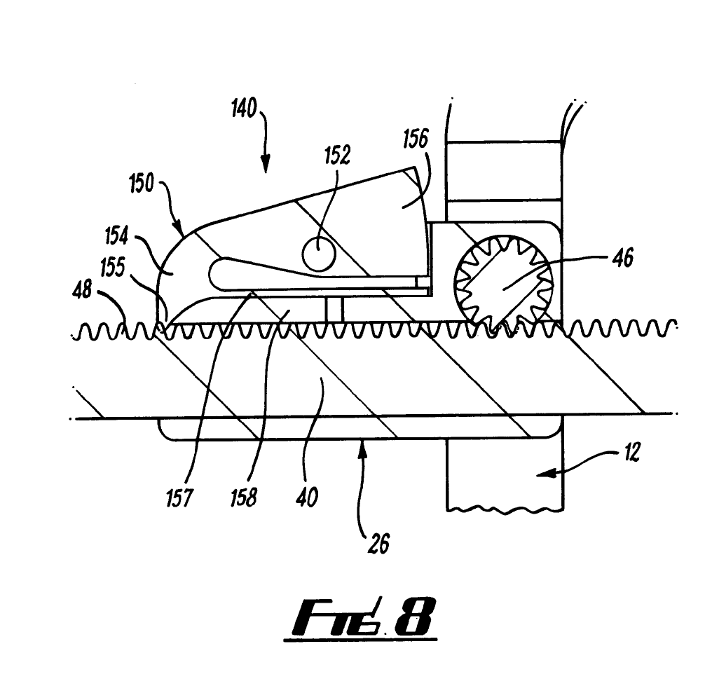

FIG. 7 is a perspective view of a second embodiment of a surgical apparatus;

FIG. 8 is a sectional view through the mounting arrangement shown in FIG. 7 showing the rocker assembly; and

FIG. 9 is a perspective view of a third Hohman retractor

Referring to FIG. 1, there is shown a first embodiment of a surgical apparatus 10 comprising a support member in the form of a generally square frame 12 comprising first and second opposite sides 14, 16 and third and fourth opposite sides 18, 20. The third and fourth opposite sides 18, 20 connect the first and second opposite sides 14, 16 to each other.

A first retractor 22 is mounted on the first side 14, and a second retractor 24 is mounted on the second side 16.

A first mounting arrangement 26 mounts the first retractor 22 on the first side 14, and a second mounting arrangement 28 mounts the second retractor 24 on the second side 16.

Referring to FIG. 1A there is shown a side view of the support member 12 in which the first side member 14 faces out of the paper. The second side member 16 and the third and fourth side members 18, 20 are hidden behind the first side member 14. The first side member 12 comprises a central region 30 and angled end regions 32, 34 which extend from the central region 30 at an angle A of substantially 30°. It will be appreciated that the second side member 16 also includes a central region 30 and first and second end regions 32, 34 extending at an angle of substantially 30° to the central region 30.

The feature of the end regions 32, 34 extending at an angle to the central region 30 of each of the first and second side members 14, 16 provides the advantage in this embodiment that the support member 12 follows the contours of the body on which the operation is effected.

The support member 12 defines a generally square central space 36 via which the operation can be carried out.

The first mounting arrangement 26 comprises a displacement member in the form of an elongate rack 40. The elongate rack 40 extends through a housing 42 fixedly mounted on the first side 14. A pinion 44, shown in broken lines is provided in the housing 42, and connected to a wheel 46 by means of which the pinion 44 can be rotated.

The rack 40 is provided with a plurality of teeth 48 arranged adjacent one another along its length, and the pinion 44 includes a plurality of corresponding teeth arranged adjacent one another around the circumference of the pinion 44.

The teeth on the pinion 44 engage the teeth 48 on the rack 40 and upon rotation of the wheel 46 the rack 40 can be moved linearly in and out of a housing 42 in the directions shown by double headed arrow B in FIG. 1. Thus, the first retractor 22 can be moved towards and away from the second retractor 24 by rotating the wheel 46.

A locking member 50 having a locking shaft 51, shown in broken lines in FIG. 1, is provided on the side of the housing 42. The locking member 50 is in the form of a bolt or screw and is threadably mounted on the housing 42. When the locking member 50 is screwed into the housing 42, it engages the rack 40 to lock the rack 40 in its position.

The first mounting arrangement 26 also includes a first securing assembly 38 to which the respective first or second retractor 22, 24 is secured. The first securing assembly 38 is shown in section in FIG. 2 and comprises a fixed member 52 through which a fastener in the form of a bolt 54 extends. The bolt 54 comprises a shaft 56, extending through the fixed member 52, and a head 58 to enable the bolt 54 to be rotated. The shaft 56 is provided with threads 60 along at least a part of its length along the end region opposite the head 58.

A movable member 62 threadably engages the shaft 56 such that when the bolt 54 is screwed into the fixed member 52, the fixed member 52 is tightened against the movable member 62. The movable member 62 comprises a generally rectangular outer profile 64 which corresponds to the inner profile of a securing element 66 of the retractor 22, as explained below.

Referring to FIGS. 3 and 4, there is shown examples of the first and second retractors 22, 24. It will be appreciated that either of the retractors 22, 24 could be used on either of the mounting arrangements 26, 28. Each of the retractors 22, 24 comprises a retractor blade 66 and a securing element 68 having a square internal profile 70.

The square internal profile 70 of the securing element 68 allows it to be slidably engaged on the square outer profile of the movable member 62 thereby preventing rotation of the retractor relative to the movable member 62.

The movable member 62 and the fixed member 52 are provided with respective interlocking formations 72 in the forms of serrations, to prevent relative rotation of the movable member 62 and the fixed member 52 when they are engaged with each other.

The blades 66 of the first and second retractors 22, 24 extend longitudinally from the securing element 68 and are curved around the longitudinal axis of the blade 66. This provides a convex surface for engagement against soft tissue in the body, thereby reducing damage to such tissue.

Referring back to FIGS. 1 and 2, when the bolt 54 tightens the movable member 62 against the fixed member 52, the interlocking formations 72 interlock with one another thereby preventing relative rotation.

Urging means in the form of a spring 74 urges the bolt 54 in the direction indicated by the arrow C in FIG. 2, thereby urging the movable member 62 against the fixed member 52. Thus, by unscrewing the bolt 54, the movable member 62 can be moved away from the fixed member 52 thereby disengaging the interlocking formations 72 from each other and allowing pivotal movement of the movable member 62 relative to the fixed member 52. This allows the retractor 22 to be pivoted and, thus, positioned at the appropriate angle.

The second retractor 24 is mounted on a second mounting arrangement 28. The second mounting arrangement 28 comprises the securing assembly 38.

The second mounting arrangement 28 comprises a second securing assembly 39 which consists of the same features and operates in the same way as the first securing assembly 38 of the first mounting arrangement 26.

The second securing assembly 39 differs from the first securing assembly 38 in that the second securing assembly 39 is fixedly attached to the second side 16 by an elongate bar 80 extending therefrom. The bar 80 is fixedly attached to the second side 16 of the support member 12 by suitable bolts 82.

The third and fourth sides of 80, 20 of the support member 12 are provided with outwardly facing serrations 84 which allow a holding member 86 to be mounted in a desired position therealong. The holding member 86 comprises upstanding projections 88 to which a chain 90 can be secured.

The chain 90 is attached to the respective Hohman retractor 100, as shown in FIG. 5.

FIG. 5 shows two Hohman retractors, which could be used in surgery. Each of the Hohman retractors 100 comprises a main portion 102 and a soft tissue engaging portion 104 extending from the main portion 102. At the opposite end of the Hohman retractors 100 there is provided a securing portion 106 of a J shape having at its free end a securing part 108 to which the chain 90 can be attached to secure the Hohman retractor 102 to the holding member 86.

First removable handles 110 are provided on each of the first and second retractors 22, 24, to pivotally manipulate the retractors 22, 24 to the desired position.

Second removable handles 112 can be provided on the Hohman retractors 100 to position the Hohman retractors 100 in the desired position.

In use, the support member 12 is arranged over the hip of the patient undergoing the operation, and an incision is made through the space 36 defined by the support member 12.

The use of the present embodiment provides the advantage that the incision need only be two or three centimetres in length, rather than, as with prior art surgical procedures ten to fifteen centimetres in length.

Suitable first and second retractors 22, 24 are mounted on the first and second mounting arrangements 26, 28 to retract the soft tissue while the initial stages of the operation take place. In order to expose the acetabulum, the first and second retractors 22, 24, having longer blades, are provided to carry out the second stage of the surgical process.

The soft tissue surrounding the acetabulum can be retracted by the use of the Hoh man retractors 102.

As can be seen from FIGS. 3 and 4 the first and second retractor 22, 24 are provided with spikes or serrations, or other formations at the free end to engage the various parts of the body being operated upon and hold such parts in place during the operation.

There is thus described a simple and effective surgical apparatus which can be used in hip replacement or resurfacing surgery allowing minimally invasive hip surgery to be carried out thereby reducing the recovery time of the patient.

Various modifications can be made without departing from the scope of the invention. For example, the handles used on the first and second retractors 22, 24 and on the Hohman retractors 102 could be of a different shape.

Referring to FIG. 7, there is shown a second embodiment of a surgical apparatus 10, which comprises most of the features of the first embodiment shown in FIG. 1 and described above. In FIG. 7, these features have been designated with the same reference numerals as in FIG. 1, and operate in the same way as the corresponding features shown in FIG. 1.

The chains 90 securing the Hohman retractors 100 to the frame 12 are omitted from FIG. 7 for clarity.

The second embodiment of the surgical apparatus 10 shown in FIG. 7 differs from the first embodiment shown in FIG. 1 in that the locking member is omitted. Instead, a rocker assembly 140 is provided, which comprises depressible member in the form of a rocker button 150. The rocker assembly 140 is shown in more detail in FIG. 8

The rocker button 150 is pivotally mounted in the housing 42 by a pivot member 152. The rocker button 150 has a first region 154 on one side of the pivot, and a second region 156 on the opposite side of the pivot 152. The first region 154 includes an engaging element 155 to engage the teeth 48 of the rack 40, as shown in FIG. 8. In FIG. 8, the rocker button 150 is in its locking position, preventing any movement of the rack 40 by the pinion 46.

An urging arrangement in the form of a leaf spring 157 is provided integrally on the rocker button 150. The leaf spring 157 extends from the first region 154 of the rocker button 150 along a reaction portion 158 of the first mounting arrangement 26.

When the rocker button 150 is depressed by pressing on the second region 156, the engaging element 155 is pivoted away from the teeth 48 of the rack 40, thereby disengaging the engaging element from the teeth 48 and moving the rocker button 150 to a release position.

The leaf spring 157 pushes against the reaction portion 158 and urges the rocker button to the locking position. Thus, when the user lets go of the rocker button 150, the leaf spring 157 pushes against the reaction portion 158 to urge the rocker button 150 towards the locking position to engage the teeth 48.

FIG. 9 shows a third retractor 160, which comprises the securing element 68 having the square internal profile 70. The third retractor 160 also includes a blade 66. A spike 162 extends from the securing element 68 along the blade 66 to project therefrom at a projecting region 164. The spike 162 terminates at its free end at a point 166.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1-50. (canceled)

51. A surgical apparatus comprising a support member having first and second opposite sides; first and second retractors on the support member; a first mounting arrangement on the first side, and a second mounting arrangement on the second side; the first mounting arrangement being configured to mount the first retractor on the first side, wherein at least the first retractor is movable towards and away from the second retractor, and the second mounting arrangement being configured to mount the second retractor on the second side; and each mounting member comprising a securing assembly to secure the first and second retractors to the first and second mounting arrangements respectively; characterised in that:
each securing assembly comprises: a fixed member; a movable member to which the first or second retractor can be secured; a fastener extending through the fixed member; and urging means on the fastener to urge the movable member into engagement with the fixed member.

52. A surgical retractor according to claim 51, wherein the movable member is threaded to threadably engage the shaft, and the movable member has a securing formation to secure the first or second retractor onto the movable member, and the fastener comprises a head and a shaft having a threaded region

53. A surgical apparatus according to claim 51, wherein the first and second retractors face each other, and the first and second retractors are pivotally movable relative to the support member.

54. A surgical apparatus according to claim 51, wherein the support member defines an access region through which surgery can be effected, the support member surrounding the aforesaid access region, and the retractors extending through the access region.

55. A surgical apparatus according to according to claim 51, wherein each of the first and second sides comprises a central region and opposite end regions extending transverse to the central region, the opposite end regions of each of the first and second sides being angled relative to the central region at an angle in the range of 20° to substantially 30° to the central region.

56. A surgical apparatus according to claim 55, wherein the support member comprises opposite third and fourth sides, the third and fourth sides extending between the first and second sides to connect the first side to the second side, thereby providing a generally rectangular profile of the support member, the third and fourth sides each including a movable holding member to secure a respective surgical member thereto, and the third and fourth sides further including serrations to hold the holding member in a selected position on the respective third or fourth side.

57. A surgical apparatus according to claim 56, wherein each holding member includes a securing formation to which a connecting element can be attached to connect the respective surgical member to the holding member, the securing formation comprising an upstanding projection, and the connecting element is elongate.

58. A surgical apparatus according to claim 57, wherein each surgical member comprises a respective manipulating member to move the respective surgical member to its desired position.

59. A surgical apparatus according to claim 51, wherein the movable member has interlocking formations, and the fixed member has corresponding interlocking formations to co-operatively engage the interlocking formations on the movable member.

60. A surgical apparatus according to claim 51, wherein the movable member includes a securing formation to secure the respective first or second retractor onto the movable member, the first and second retractors each comprising a securing element slidably mounted on the securing formation to engage the respective movable member, and wherein the securing formation and the securing element are correspondingly configured to substantially prevent relative rotation of the movable member and the respective retractor.

61. A surgical apparatus according to claim 60, wherein the securing formation comprises a generally rectangular outer profile of the movable member, and the securing element having a generally rectangular inner profile, said inner profile of the securing element corresponding to the outer profile of the securing formation.

62. A surgical apparatus according to claim 51, wherein each of the movable members is pivotally movable relative to the respective first or second fixed members to arrange the respective first or second retractors at a desired angle relative to the support member.

63. A surgical apparatus according to claim 51, wherein the first mounting arrangement comprises a displacement mechanism to move the first retractor towards and away from the second retractor, the displacement mechanism comprising a displacement member to which the respective fixed member is attached, the displacement member being elongate and comprisesing a rack, and the displacement mechanism further including a housing, through which the displacement member movably extends.

64. A surgical apparatus according to claim 63, wherein the first mounting arrangement includes a driver to drive the displacement member, the driver comprising a rotatable head and a cog in engagement with the displacement member.

65. A surgical apparatus according to claim 63, wherein a locking member is provided to lock the displacement member to the housing, the locking member comprising a threaded member threadably receivable in the housing to engage the mounting member, and the locking member further includes a rotatable head mounted on the threaded member to rotate the threaded member.

66. A surgical apparatus according to claim 63, wherein a locking member is provided to lock the displacement member to the housing, the locking member comprising a depressible element for engaging the displacement member, the depressible member being movable between a locking position and a release position by pressing thereon.

67. A surgical apparatus according to claim 66, wherein the depressible element can pivot about a pivot member between the locking and the release positions, the depressible element being depressible at a first region on one side of the pivot member to move the depressible element to the locking position, and the depressible element is depressible at a second region on the opposite side of the pivot member to move the depressible element to the release position.

68. A retractor comprising a securing element for securing the retractor to a support member and a blade extending from the securing element.

69. A retractor according to claim 68, wherein the securing element comprises an inner generally rectangular profile for co-operation with a correspondingly shaped securing part on the support member, and the blade has a longitudinal axis and is curved about the longitudinal axis.

70. A method of using a surgical apparatus as claimed in claim 51, said method comprising arranging the first and second retractors on the support member, inserting the retractors in an incision in a body and moving the first retractor towards and/or away from the second retractor to retract a portion of the body upon which surgery is being effected.

71. A method according to claim 70, comprising pivoting the first retractor relative to the support member, and pivoting the second retractor relative to the support member.

72. A method according to claim 70, comprising arranging the first and second retractors in opposed positions relative to each other, and linearly moving the first retractor towards and/or away from the first retractor.

73. A method according to claims 70, wherein the support member defines an access region, and the method comprises effecting the surgery through the access region.

74. A method according to claim 70, comprising mounting the retractors on first and second opposite sides of the support member, and securing surgical members to a respective one of third and fourth opposite sides of the support member, wherein each surgical member comprises a Hohman retractor.
